(19) United States
(12) Patent Application Publication
(10) Pub. No.: US 2006/0057587 A1
(43) Pub. Date: Mar. 16, 2006

(54) Oligonucleotide arrays comprising background probes

(76) Inventors: Scott J. Vacha, The Woodlands, TX (US); Eric M. Leproust, San Jose, CA (US)

Correspondence Address: AGILENT TECHNOLOGIES, INC.;INTELLECTUAL PROPERTY ADMINISTRATION, LEGAL DEPT., P.O. BOX 7599, M/S DL429, LOVELAND, CO, 80537-0599, US

(21) Appl. No.: 10/939,650

(22) Filed: Sep. 13, 2004

(51) Int. Cl. C12Q 1/68; C12M 1/34

(52) U.S. Cl. 435/6; 435/287.2

(57) ABSTRACT

The invention relates to chemical arrays comprising a plurality of features comprising biopolymers at known locations on the substrate, wherein at least one feature comprises specific hybridization probes designed or selected to bind to specific target sequences and background probes for evaluating non-specific binding of target. Methods, systems, and kits for making and using the arrays are also described.

### BACKGROUND

Chemical arrays have become an increasingly important tool in the biotechnology industry and related fields. These arrays, comprising a plurality of chemical species (e.g., biopolymers) arranged on a solid support surface in the form of an array or pattern of features, find use in a variety of applications, including gene expression analysis, drug screening, nucleic acid sequencing, mutation analysis, and the like.

Biopolymer arrays can be fabricated by depositing biopolymers on a support by or in situ synthesis methods. Deposition methods basically involve depositing biopolymers at predetermined locations on a substrate that is suitably activated such that the biopolymers can link to the substrate. Biopolymers of different sequence may be deposited at difference regions on the substrate to yield the completed array. Typical procedures known in the art for deposition of previously obtained polynucleotides, particularly DNA, such as whole oligomers or cDNA, are to load a small volume of DNA in solution in one or more drop dispensers such as the tip of a pin or in an open capillary and, touch the pin or capillary to the surface of the substrate. Such a procedure is described in U.S. Pat. No. 5,807,522. When the fluid touches the surface, some of the fluid is transferred. The pin or capillary must be washed prior to picking up the next type of DNA for spotting onto the array. This process is repeated for many different sequences and, eventually, the desired array is formed. Alternatively, the DNA can be loaded into a drop dispenser in the form of a pulse jet head and fired onto the substrate. Such a technique has been described in WO 95/25116 and WO 98/41531, and elsewhere.

In situ synthesis methods include those described in U.S. Pat. No. 5,449,754 for synthesizing peptide arrays, as well as WO 98/41531 and the references cited therein for synthesizing polynucleotides (specifically, DNA) using phosphoramidite or other chemistry. Additional patents describing in situ nucleic acid array synthesis protocols and devices include U.S. Pat. Nos. 6,451,998; 6,446,682; 6,440,669; 6,420,180; 6,372,483; 6,323,043; and 6,242,266; the disclosures of which patents are herein incorporated by reference.

Biopolymer arrays can provide a plurality of different probe molecules, enabling high throughput evaluation of a sample. In array-based assays, the array surface is typically contacted with one or more targets, such as a sample of polynucleotides, proteins, peptides, small molecules and the like, under conditions that promote specific, high-affinity binding of target molecules to one or more array features comprising these probes. Generally, the goal is to identify one or more position-addressable features of the array that bind to a target, for example to detect, quantify and/or characterize the sequence of a target molecule.

Typically, target molecules are labeled with a detectable label such as a fluorescent molecule, so that the formation of probe-target complexes may be detected by virtue of the fluorescence of the label. The detection of binding complexes may be used to obtain information about the nature and/or amount of target biomolecules in the solution. For example, quantification of the level of fluorescence associated with a bound probe represents a direct measurement of the level of binding of the probe to the target. In turn, this measurement of binding represents an estimate of the abundance of a particular target in the sample.

Binding complexes may be detected by reading or scanning the array with, for example, optical means, although other methods may also be used, as appropriate for the particular assay. For example, laser light may be used to excite fluorescent labels attached to the targets, generating a signal only in those spots on the array that have a labeled target molecule bound to a probe molecule. This pattern may then be digitally scanned for computer analysis. Such patterns can be used to generate data for biological assays such as gene expression analysis, identification of drug targets, single-nucleotide polymorphism mapping, monitoring samples from patients to track their response to treatment, assessing the efficacy of new treatments, etc.

Multiple approaches currently exist to subtract background signal resulting from non-specific binding of a target to one or more features on an array when performing data analysis. The use of local background on an in situ-synthesized array does not properly model the non-specific binding that can occur on DNA that is bound to glass. Because the spatial non-uniformity of background pixel intensities (i.e., non-feature intensities) may exist on a array image, using these pixels for local background subtraction may lead to the non-random spatial distribution of differentially expressed genes on an array (based on statistical methods). A second approach for background subtraction involves the use of independent negative control features (DNA) randomly distributed throughout the array. While the intensity values of these negative control features may allow for better estimates of non-specific binding and allow for a solution to spatial non-uniformity of differentially expressed genes, the number of necessary negative control features may not be practical given the available printing positions on an in situ-synthesized array.

### SUMMARY OF THE INVENTION

In one embodiment, the invention provides a method for making a chemical array. The method comprises arraying a plurality of biopolymers on a substrate, thereby forming a plurality of features comprising biopolymers at known locations on the substrate. In one aspect, at least one feature comprises a set of specific hybridization probes designed or selected to bind to specific target sequences and a set of background probes for evaluating non-specific binding of target.

Biopolymers forming features may be deposited on the substrate in a variety of ways. For example, biopolymers, such as nucleic acids (e.g., DNA) may be deposited on a substrate at a plurality of known locations. Biopolymers also can be synthesized in situ by iteratively depositing nucleotide monomers at discrete, known locations on the substrate, e.g., by using a pulse jet printer.

In one aspect, background probes are enriched for at locations within a feature, e.g., a within-feature background probe is surrounded by more background probes than specific hybridization probes. Background probes may be clustered. For example, in one aspect, the background probes are located at the perimeter of a feature while the specific hybridization probes are located in the center of the feature.

In one embodiment, the specific hybridization probes are, on average, longer than the background probes. In one aspect, the specific hybridization probes are 60 base pairs or greater. However, specific hybridization probes may be less than about 60 base pairs and in some aspects, specific hybridization probes are about 25 base pairs.

In one aspect, background probes comprise at least about 5 base pairs, at least about 10 base pairs, at least about 15 base pairs, or at least about 20 base pairs. In another aspect, background probes comprise a subsequence of the sequence of a specific hybridization probe. In a further aspect, specific hybridization probes comprise a sequence that is substantially identical to the background probe sequence and a target identifying sequence which is sufficiently complementary to a target sequence to specifically bind to the target sequence under stringent conditions. In one aspect, the background probe is not sufficiently complementary to the target sequence identified by the specific hybridization probe to selectively bind to the target sequence under stringent conditions, i.e., the background probe does not exhibit any sequence specificity for the target sequence, and if it binds to any extent, it will bind with equal likelihood to other sequences in a sample.

As discussed above, specific hybridization probes may be formed at a spot on an array by iteratively depositing nucleotide monomers at the spot to form a biopolymer. The spot of biopolymers formed defines a feature on the array. In one embodiment, specific hybridization probes are formed by iteratively depositing nucleotide monomers at a location within a feature to form a biopolymer of n base pairs, while limiting biopolymer formation at another location within the feature to form a biopolymer of fewer than n base pairs. In one aspect, nucleotide monomers are deposited in droplets of decreasing volume at the feature. In another aspect, nucleotide monomers are deposited in droplets by a droplet dispenser and either or both the droplet dispenser or substrate is moved after a selected number of rounds of synthesis so that the droplet falls on a location of the feature to comprise specific hybridization probes, continuing synthesis at that location, and does not fall on a location of the feature to comprise background probes (i.e., limiting synthesis at that location). In another aspect, specific hybridization probes are formed by iteratively depositing monomer to form a biopolymer of n base pairs and background probes are formed by selectively blocking biopolymer synthesis when the probes have reached a length that is fewer than n base pairs. In another aspect, after a selected number of rounds of synthesis, specific hybridization probes are selectively activated and/or selectively deprotected, allowing synthesis at the specific hybridization probes while limiting synthesis of background probes which are not activated and/or not deprotected.

In one embodiment, the invention provides systems for forming a chemical array comprising a plurality of features, at least one feature comprising specific hybridization probes and background probes. In one aspect, the system comprises a substrate holder, a head comprising one or more dispensing units for dispensing a drop of fluid on a substrate in the substrate holder and a transporter for moving either, or both, the substrate or dispensing chamber(s) relative to one another. In another aspect, the system further comprises a processor for controlling dispensing parameters, such as movement of the substrate and/or head and/or deposition of drops (e.g., volume of drops, agents within drops, drop velocity, drop viscosity, and the like) from the one or more dispensing chambers, to selectively limit synthesis of background probes within a feature while allowing synthesis of specific hybridization probes. In still a further embodiment, the invention provides a method for ordering a chemical array comprising at least one feature comprising background probes and specific hybridization probes, comprising providing instructions to the processor of the system, specifying a pattern of drops to be dispensed on a substrate. The pattern may include the nature of nucleotide monomers deposited, locations of features on the array, and instructions for controlling dispensing parameters to control the synthesis of background probes and specific hybridization probes at one or more features on the array.

In one embodiment, the invention also provides a chemical array formed by any of the above methods and/or systems.

In one aspect, the invention provides a chemical array comprising a plurality of features. Each feature comprises a plurality of specific hybridization probes and background probes. In one aspect, background probes comprise at least about 5%, at least about 10%, at least about 15%, at least about 20%, at least about 30%, at least about 40% or at least about 50% of the probes at a feature.

In another aspect, the invention provides a chemical array comprising a plurality of features, wherein each feature comprises a plurality of specific hybridization probes and background probes, and wherein the background probe sequences comprise a subsequence of the specific hybridization probes and background probes within at least two features are substantially identical (e.g., at least about 95%, or at least about 99% identical as determined using algorithms known in the art (e.g., such as BLAST) and standard default parameters). In one aspect, each feature of the plurality comprises
substantially identical background probes.

In a further aspect, the invention provides a substrate comprising a plurality of chemical arrays, at least one array being a chemical array as discussed above comprising at least one feature comprising specific hybridization probes and background probes.

The invention further provides a method for detecting specific binding of a target molecule in a sample to a probe. The method comprises providing an array comprising a plurality of biopolymer features arrayed at known locations on a substrate, wherein at least one of the features comprises a plurality of specific hybridization probes and a plurality of background probes. In one aspect, a plurality of features comprises specific hybridization probes and background probes. An amount of binding of target to a feature is determined and then adjusted based on the amount of binding of target to within-feature background probes in the feature. In one aspect, binding to within-feature background probes is identified based on the location of target-probe complexes at the feature (e.g., such as determined by the intensity of a fluorescent signal measured at a location of the feature known to contain background probes).

In another embodiment, the invention also provides a computer program product comprising instructions for forming a pattern of features on an array using a system described above, at least one feature comprising specific hybridization probes designed or selected to bind to specific target sequences and background probes for evaluating non-specific binding of target.

In still another embodiment, the invention provides a method for evaluating non-specific background signal at a feature on a chemical array which comprises background probes and specific hybridization probes. The method comprises positioning a template on an image or representation of an array that is predicted to have a feature deposited or written thereof, wherein the template conforms in dimensions to an area of a feature comprising background probes, and excludes an area of the feature comprising specific hybridization probes. Feature information (e.g., such as pixel intensity) is then extracted from the template area.

In a further embodiment, the invention provides a method for receiving data for a feature on a chemical array comprising background probes and specific hybridization probes. The method may be used to determine the presence and/or amount of a target molecule that specifically binds to the specific hybridization probe and thus the presence and/or amount of target in a sample.

### DETAILED DESCRIPTION

In one aspect, the invention provides an array comprising a plurality of features, each feature comprising a specific hybridization probe designed or selected to bind to specific target sequences and a background probe provided as a negative control to evaluate non-specific binding of target sequences to an array.

Before the present invention is described, it is to be understood that this invention is not limited to particular embodiments described, as such may, of course, vary. It is also to be understood that the terminology used herein is for the purpose of describing particular embodiments only, and is not intended to be limiting, since the scope of the present invention will be limited only by the appended claims.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, the preferred methods and materials are now described. Methods recited herein may be carried out in any order which is logically possible, in addition to a particular order disclosed.

All publications mentioned herein are incorporated herein by reference to disclose and describe the methods and/or materials in connection with which the publications are cited.

The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates which may need to be independently confirmed.

The practice of the present invention will employ, unless otherwise indicated, conventional techniques of chemistry, biochemistry, molecular biology, and medicine, including diagnostics, which are within the skill of the art. Such techniques are explained fully in the literature. See, e.g., Solid-Phase Synthesis, Blossey, E. C. and Neckers, D. C. Eds. 1975; Sambrook, Fritsch & Maniatis, Molecular Cloning: A Laboratory Manual; DNA Cloning, Vols. I and II (D. N. Glover ed.); Oligonucleotide Synthesis (M. J. Gait ed.); Nucleic Acid Hybridization (B. D. Hames & S. J. Higgins eds.); and the series, Methods In Enzymology (S. Colowick and N. Kaplan eds., Academic Press, Inc.); Beaucage and Carruthers, Tetrahedron Lett., 22:1859-1862 (1981); Matteucci, et al, J. Am. Chem. Soc., 103:3185 (1981); Letsinger, R. L. and Mahadevan, V., J. Amer. Chem. Soc., 88:5319-5324.

The following definitions are provided for specific terms that are used in the following written description.

It must be noted that as used herein and in the appended claims, the singular forms “a”, “and”, and “the” include plural referents unless the context clearly dictates otherwise. Thus, for example, reference to “a laser” includes a plurality of such lasers and reference to “the array” includes reference to one or more arrays and equivalents thereof known to those skilled in the art, and so forth.

Where a range of values is provided, it is understood that each intervening value, to the tenth of the unit of the lower limit unless the context clearly dictates otherwise, between the upper and lower limit of that range and any other stated or intervening value in that stated range is encompassed within the invention. The upper and lower limits of these smaller ranges may independently be included in the smaller ranges is also encompassed within the invention, subject to any specifically excluded limit in the stated range. Where the stated range includes one or both of the limits, ranges excluding either both of those included limits are also included in the invention.

It will also be appreciated that throughout the present application, that words such as “cover”, “base” “front”, “back”, “top”, “upper”, and “lower” are used in a relative sense only.

A “biopolymer” is a polymer of one or more types of repeating units. Biopolymers are typically found in biological systems and particularly include polysaccharides (such as carbohydrates), peptides (which term is used to include polypeptides and proteins) and polynucleotides as well as their analogs such as those compounds composed of or containing amino acid analogs or non-amino acid groups, or nucleotide analogs or non-nucleotide groups. Biopolymers include polynucleotides in which the conventional backbone has been replaced with a non-naturally occurring or synthetic backbone, and nucleic acids (or synthetic or naturally occurring analogs) in which one or more of the conventional bases has been replaced with a group (natural or synthetic) capable of participating in Watson-Crick type hydrogen bonding interactions. Polynucleotides include single or multiple stranded configurations, where one or more of the strands may or may not be completely aligned with another. A “nucleotide” refers to a sub-unit of a nucleic acid and has a phosphate group, a 5 carbon sugar and a nitrogen containing base, as well as functional analogs (whether synthetic or naturally occurring) of such sub-units which in the polymer form (as a polynucleotide) can hybridize with naturally occurring polynucleotides in a sequence specific manner analogous to that of two naturally occurring polynucleotides. Biopolymers include DNA (including cDNA), RNA, oligonucleotides, and PNA and other polynucleotides as described in U.S. Pat. No. 5,948,902 and references cited therein (all of which are also incorporated herein by reference), regardless of the source. An “oligonucleotide” generally refers to a nucleotide multimer of about 10 to 100 nucleotides in length, while a “polynucleotide” includes a nucleotide multimer having any number of nucleotides. A “biomonomer” references a single unit, which can be linked with the same or other biomonomers to form a biopolymer (e.g., a single amino acid or nucleotide with two linking groups one or both of which may have removable protecting groups).

As used herein, the terms “nucleic acid molecule,” “oligonucleotide,” “nucleotide sequence” and “polynucleotide” may be used interchangeably, and refer to nucleic acid molecules and polymers thereof, including conventional purine or pyrimidine bases as well as base analogs. Such molecules include without limitation, nucleic acids, and fragments thereof, from any source, synthetic or natural, in purified or unpurified form including DNA, double-stranded or single stranded (dsDNA and ssDNA), and RNA, including t-RNA, m-RNA, r-RNA, mitochondrial DNA and RNA, chloroplast DNA and RNA, DNA/RNA hybrids, or mixtures thereof, genes, chromosomes, plasmids, the genomes of biological materials such as microorganisms, e.g. bacteria, yeasts, viruses, viroids, molds, fungi, plants, animals, humans, and the like; polynucleotides containing an N- or a C-glycoside of a purine or pyrimidine base; other polymers containing nonnucleotidic backbones, for example, abasic phosphodiesters, polyamide (e.g., peptide nucleic acids (PNAs)) and polymorpholino (commercially available from the Anti-Virals, Inc., Corvallis, Oreg., as Neugene™ polymers), and other synthetic sequence-specific nucleic acid polymers providing that the polymers contain nucleobases in a configuration which allows for base pairing and base stacking, such as is found in DNA and RNA. The polynucleotide can be only a minor fraction of a complex mixture such as a biological sample. Also included are genes, such as hemoglobin gene for sickle-cell anemia, cystic fibrosis gene, oncogenes, cDNA, and the like.

The terms “polynucleotide” and “oligonucleotide,” also include known types of modifications, for example, labels which are known in the art, methylation, “caps,” substitution of one or more of the naturally occurring nucleotides with an analog, internucleotide modifications such as, for example, those with uncharged linkages (e.g., methyl phosphonates, phosphotriesters, phosphoramidates, carbamates, etc.), with negatively charged linkages (e.g., phosphorothioates, phosphorodithioates, etc.), and with positively charged linkages (e.g., aminoalklyphosphoramidates, aminoalkylphosphotriesters), those containing pendant moieties, such as, for example, proteins (including nucleases, toxins, antibodies, signal peptides, poly-L-lysine, etc.), those with intercalators (e.g., acridine, psoralen, etc.), those containing chelators (e.g., metals, radioactive metals, boron, oxidative metals, etc.), those containing alkylators, those with modified linkages (e.g., alpha anomeric nucleic acids, etc.), as well as unmodified forms of the polynucleotide.

Various techniques can be employed for preparing a polynucleotide. Such polynucleotides can be obtained by biological synthesis or by chemical synthesis. For short sequences (up to about 100 nucleotides), chemical synthesis is economical, provides a convenient way of incorporating low molecular weight compounds and/or modified bases during specific synthesis steps, and is very flexible in the choice of length and region of target polynucleotide binding sequence. Polynucleotides can be synthesized by standard methods such as those used in commercial automated nucleic acid synthesizers. Chemical synthesis of DNA on a suitably modified glass or resin can result in DNA covalently attached to the surface, potentially advantageous in washing and sample handling. For longer sequences standard replication methods employed in molecular biology can be used such as the use of M13 for single stranded DNA as described by Messing, J., Methods Enzymol., 1983, 101:20-78; or the use of polymerase chain reaction as described in U.S. Pat. Nos. 4,683,195; 4,683,202 and 4,965,188.

As used herein, the term “substantially identical” refers to nucleic acid molecules comprising at least about 95%, or at least about 99% identity at the nucleotide sequence level. The terms “percent identity” refers to the percentage of residue matches between at least two polynucleotide sequences aligned using a standardized algorithm. Such an algorithm may insert gaps in the sequences being compared in order to optimize alignment between two sequences, and therefore achieve a more meaningful comparison of the two sequences. For example, the Basic Local Alignment Search Tool (BLAST) (Altschul, S. F. et al. (1990) J. Mol. Biol. 215:403-410), which is available at http://www.ncbi.nlm.nih.gov/BLAST/, for example. The BLAST software suite includes various sequence analysis programs including “blastn,” that is used to align a known polynucleotide sequence with other polynucleotide sequences from a variety of databases. Also available is a tool called “BLAST 2 Sequences” that is used for direct pairwise comparison of two nucleotide sequences. “BLAST 2 Sequences” can be accessed and used interactively at http://www.ncbi.nlm.nih.gov/gorf/b12.html. The “BLAST 2 Sequences” tool can be used for both blastn and blastp (discussed below). BLAST programs are commonly used with gap and other parameters set to default settings. For example, to compare two nucleotide sequences, one may use blastn with the “BLAST 2 Sequences” tool Version 2.0.12 (Apr. 21, 2000) set at default parameters. Such default parameters may be, for example: matrix: BLOSUM62; reward for match: 1; penalty for mismatch: −2; Open Gap: 5 and Extension Gap: 2 penalties; Gap×drop-off: 50; Expect: 10; Word Size: 11; Filter: on. Percent identity may be measured over the length of an entire defined sequence, e.g., the length of a background probe, for example.

The term “substrate” as used herein refers to a surface upon which biopolymer molecules or probes, e.g., an array, may be adhered.

As used herein an “array,” includes any two-dimensional or substantially two-dimensional (as well as a three-dimensional) arrangement of addressable regions bearing a particular chemical moiety or moieties (e.g., biopolymers such as polynucleotide or oligonucleotide sequences (nucleic acids), polypeptides (e.g., proteins), carbohydrates, lipids, etc.) associated with that region. In the broadest sense, the preferred arrays are arrays of polymeric binding agents, where the polymeric binding agents may be any of: polypeptides, proteins, nucleic acids, polysaccharides, synthetic mimetics of such biopolymeric binding agents, etc. In many embodiments of interest, the arrays are arrays of nucleic acids, including oligonucleotides, polynucleotides, cDNAs, mRNAs, synthetic mimetics thereof, and the like. Where the arrays are arrays of nucleic acids, the nucleic acids may be covalently attached to the arrays at any point along the nucleic acid chain, but are generally attached at one of their termini (e.g. the 3′ or 5′ terminus). Sometimes, the arrays are arrays of polypeptides, e.g., proteins or fragments thereof.

Any given substrate may carry one, two, four or more or more arrays disposed on a front surface of the substrate. Depending upon the use, any or all of the arrays may be the same or different from one another and each may contain multiple spots or features. A typical array may contain more than ten, more than one hundred, more than one thousand more ten thousand features, or even more than one hundred thousand features, in an area of less than 20 cm² or even less than 10 cm². For example, features may have widths (that is, diameter, for a round spot) in the range from aboout 10 μm to 1.0 cm. In other embodiments each feature may have a width in the range of about 1.0 μm to 1.0 mm, usually about 5.0 μm to 500 μm, and more usually 10 μm to 200 μm. Non-round features may have area ranges equivalent to that of circular features with the foregoing width (diameter) ranges. At least some, or all, of the features are of different compositions (for example, when any repeats of each feature composition are excluded the remaining features may account for at least 5%, 10%, or 20% of the total number of features).

All of the features may be different, or some could be the same (for example, when any repeats of each feature composition are excluded the remaining features may account for at least 5%, 10%, or 20% of the total number of features). In some aspects, features are arranged in straight line rows extending left to right. In the case where arrays are formed by the in situ or deposition of previously obtained biopolymers by depositing for each feature a droplet of reagent in each cycle such as by using a pulse jet such as an inkjet type head, interfeature areas will typically be present which do not carry any polynucleotide or moieties of the array features. It will be appreciated though, that the interfeature areas could be of various sizes and configurations. It will also be appreciated that there need not be any space separating arrays in a multi-array substrate from one another although there typically will be. As per usual, A, C, G, T represent the usual nucleotides. It will be understood that there may be a linker molecule (not shown) of any known types between the front surface and the first nucleotide.

Interfeature areas will typically (but not essentially) be present which do not carry any polynucleotide (or other biopolymer or chemical moiety of a type of which the features are composed). Such interfeature areas typically will be present where the arrays are formed by processes involving drop deposition of reagents but may not be present when, for example, photolithographic array fabrication processes are used. It will be appreciated though, that the interfeature areas, when present, could be of various sizes and configurations.

Each array may cover an area of less than 100 cm², or even less than 50 cm², 10 cm² or 1 cm². In many embodiments, the substrate carrying the one or more arrays will be shaped generally as a rectangular solid (although other shapes are possible), having a length of more than 4 mm and less than 1 m, usually more than 4 mm and less than 600 mm, more usually less than 400 mm; a width of more than 4 mm and less than 1 m, usually less than 500 mm and more usually less than 400 mm; and a thickness of more than 0.01 mm and less than 5.0 mm, usually more than 0.1 mm and less than 2 mm and more usually more than 0.2 and less than 1 mm. With arrays that are read by detecting fluorescence, the substrate may be of a material that emits low fluorescence upon illumination with the excitation light. Additionally in this situation, the substrate may be relatively transparent to reduce the absorption of the incident illuminating laser light and subsequent heating if the focused laser beam travels too slowly over a region. For example, a substrate may transmit at least 20%, or 50% (or even at least 70%, 90%, or 95%), of the illuminating light incident on the front as may be measured across the entire integrated spectrum of such illuminating light or alternatively at 532 nm or 633 nm.

An array is “addressable” when it has multiple regions of different moieties (e.g., different polynucleotide sequences) such that a region (i.e., a “feature” or “spot” of the array) at a particular predetermined location (i.e., an “address”) on the array will detect a particular target or class of targets (although a feature may incidentally detect non-targets of that feature). Array features are typically, but need not be, separated by intervening spaces.

In the case of an array, a “target” refers to a moiety in a mobile phase (typically fluid), to be detected by probes (“target probes”) which are bound to the substrate at the various regions. However, either of the “target” or “probe” may be the one that is to be evaluated by the other (thus, either one could be an unknown mixture of polynucleotides to be evaluated by binding with the other).

A “scan region” refers to a contiguous (preferably, rectangular) area in which the array spots or features of interest, as defined above, are found. The scan region is that portion of the total area illuminated from which resulting fluorescence may be detected and recorded. For example, a scan region may include the entire area of a substrate (such as a slide) scanned in each pass of a lens, between a first feature of interest, and a last feature of interest, even if there exist intervening areas which lack features of interest.

An “array layout” refers to one or more characteristics of the features, such as feature positioning on the substrate, one or more feature dimensions, and an indication of a moiety (e.g., such as a nucleic acid sequence) at a given location.

A “set” or “sub-set” of any item (such as a set of arrays) may contain only one of the item, or only two, or three, or any multiple number of the items. An “array”, unless a contrary intention appears, includes any one, two or three dimensional arrangement of addressable regions bearing a particular chemical moiety to moieties (for example, biopolymers such as polynucleotide sequences) associated with that region. An array is “addressable” in that it has multiple regions of different moieties (for example, different polynucleotide sequences) such that a region (a “feature” or “spot” of the array) at a particular predetermined location (an “address”) on the array will detect a particular target or class of targets (although a feature may incidentally detect non-targets of that feature).

A “remote location,” refers to location other than the location at which the array is present and hybridization occurs. For example, a remote location could be another location (e.g., office, lab, etc.) in the same city, another location in a different city, another location in a different state, another location in a different country, etc. As such, when one item is indicated as being “remote” from another, what is meant is that the two items are at least in different rooms or different buildings, and may be at least one mile, ten miles, or at least one hundred miles apart.

“Communicating information” refers to transmitting the data representing that information as signals (e.g., electrical, optical, radio, magnetic, etc) over a suitable communication channel (e.g., a private or public network).

As used herein, a component of a system which is “in communication with” or “communicates with” another component of a system receives input from that component and/or provides an output to that component to implement a system function. A component which is “in communication with” or which “communicates with” another component may be, but is not necessarily, physically connected to the other component. For example, the component may communicate information to the other component and/or receive information from the other component.

“Forwarding” an item refers to any means of getting that item from one location to the next, whether by physically transporting that item or otherwise (where that is possible) and includes, at least in the case of data, physically transporting a medium carrying the data or communicating the data.

An array “package” may be the array plus only a substrate on which the array is deposited, although the package may include other features (such as a housing with a chamber).

A “computer-based system” refers to the hardware means, software means, and data storage means used to analyze the information of the present invention. The minimum hardware of the computer-based systems of the present invention comprises a central processing unit (CPU), input means, output means, and data storage means. A skilled artisan can readily appreciate that any one of the currently available computer-based system are suitable for use in the present invention. The data storage means may comprise any manufacture comprising a recording of the present information as described above, or a memory access means that can access such a manufacture. In certain instances a computer-based system may include one or more wireless devices.

To “record” data, programming or other information on a computer readable medium refers to a process for storing information, using any such methods as known in the art. Any convenient data storage structure may be chosen, based on the means used to access the stored information. A variety of data processor programs and formats can be used for storage, e.g. word processing text file, database format, etc.

A “processor” references any hardware and/or software combination which will perform the functions required of it. For example, any processor herein may be a programmable digital microprocessor such as available in the form of a electronic controller, mainframe, server or personal computer (desktop or portable). Where the processor is programmable, suitable programming can be communicated from a remote location to the processor, or previously saved in a computer program product (such as a portable or fixed computer readable storage medium, whether magnetic, optical or solid state device based). For example, a magnetic medium or optical disk may carry the programming, and can be read by a suitable reader communicating with each processor at its corresponding station.

A “reader” refers to any device for evaluating arrays, including an array imager and an array scanner. An “array imager” captures a two-dimensional wide-field image of an array, e.g., an entire array or multi-pixel region thereof, and may employ a CCD or other detector. An “array scanner” moves a field of illumination across an array, typically in a line or series of lines, and reads light emitted from the array. In many scanners, an optical light source, particularly a laser light source, generates a collimated beam. The collimated beam is focused on the array and sequentially illuminates small surface regions of known location (i.e. a position) on an array substrate. The resulting signals from the surface regions are collected either confocally (employing the same lens used to focus the light onto the array) or off-axis (using a separate lens positioned to one side of the lens used to focus the onto the array). The collected signals are then transmitted through appropriate spectral filters, to an optical detector. A recording device, such as a computer memory, records the detected signals and builds up a raster scan file of intensities as a function of position, or time as it relates to the position. Such intensities, as a function of position, are typically referred to in the art as “pixels”. Biopolymer arrays are often scanned and/or scan results are often represented at 5 or 10 micron pixel resolution. To achieve the precision required for such activity, components such as the lasers must be set and maintained with particular alignment. Scanners may be bi-directional, or unidirectional, as is known in the art.

The scanner typically used for the evaluation of arrays includes a scanning fluorometer. A number of different types of such devices are commercially available from different sources, such as such as Perkin-Elmer, Agilent, or Axon Instruments, etc., and examples of typical scanners are described in U.S. Pat. Nos. 5,091,652; 5,760,951, 6,320,196 and 6,355,934.

The term “assessing” and “evaluating” are used interchangeably to refer to any form of measurement, and includes determining if an element is present or not. The terms “determining,” “measuring,” and “assessing,” and “assaying” are used interchangeably and include both quantitative and qualitative determinations. Assessing may be relative or absolute. “Assessing the presence of” includes determining the amount of something present, as well as determining whether it is present or absent.

The term “using” has its conventional meaning, and, as such, means employing, e.g. putting into service, a method or composition to attain an end. For example, if a program is used to create a file, a program is executed to make a file, the file usually being the output of the program. In another example, if a computer file is used, it is usually accessed, read, and the information stored in the file employed to attain an end. Similarly if a unique identifier, e.g., a barcode is used, the unique identifier is usually read to identify, for example, an object or file associated with the unique identifier.

Arrays Comprising Background Probes

In one aspect, the invention provides a chemical array comprising a plurality of features arrayed on a substrate at known locations. At least one feature in a chemical array according to the invention comprises background probes for evaluating non-specific binding target molecules in addition to specific hybridization probes designed or selected to bind to specific target sequences.

In one aspect, a background probe in a feature comprises a subsequence of a specific hybridization probe in the feature. In another aspect, the sequence of the background probe comprises a sequence that is not complementary to a sequence in a target sequence to which a specific hybridization probe would otherwise specifically hybridize. As used herein, the terms “specifically hybridizing,” “hybridizing specifically to” and “specific hybridization” and “selectively hybridize to,” are used interchangeably and refer to the binding, duplexing, complexing or hybridizing of a nucleic acid molecule preferentially to a particular nucleotide sequence under stringent conditions. “Hybridizing” and “binding”, with respect to polynucleotides, are used interchangeably.

The term “stringent assay conditions” as used herein refers to conditions that are compatible to produce binding pairs of nucleic acids, e.g., surface bound and solution phase nucleic acids, of sufficient complementarity to provide for the desired level of specificity in the assay while being less compatible to the formation of binding pairs between binding members of insufficient complementarity to provide for the desired specificity. Stringent assay conditions are the summation or combination (totality) of both hybridization and wash conditions.

“Stringent hybridization conditions” and “stringent hybridization wash conditions” in the context of nucleic acid hybridization (e.g., as in array, Southern or Northern hybridizations) are sequence dependent, and are different under different experimental parameters. The phrase “hybridizing specifically to”, refers to the binding, duplexing, or hybridizing of a molecule only to a particular nucleotide sequence under stringent conditions when that sequence is present in a complex mixture (e.g., total cellular) DNA or RNA. The term “stringent conditions” refers to conditions under which a probe will hybridize to its target subsequence, but to no other sequences. Stringent conditions are sequence-dependent and will be different in different circumstances. Longer sequences hybridize specifically at higher temperatures. Generally, stringent conditions are selected to be about 5° C. lower than the thermal melting point (Tm) for the specific sequence at a defined ionic strength and pH. The Tm is the temperature (under defined ionic strength, pH, and nucleic acid concentration) at which 50% of the probes complementary to the target sequence hybridize to the target sequence at equilibrium. (As the target sequences are generally present in excess, at Tm, 50% of the probes are occupied at equilibrium).

Stringent hybridization conditions that can be used to identify nucleic acids within the scope of the invention can include, e.g., hybridization in a buffer comprising 50% formamide, 5×SSC, and 1% SDS at 42° C., or hybridization in a buffer comprising 5×SSC and 1% SDS at 65° C., both with a wash of 0.2×SSC and 0.1% SDS at 65° C. Exemplary stringent hybridization conditions can also include a hybridization in a buffer of 40% formamide, 1 M NaCl, and 1% SDS at 37° C., and a wash in 1×SSC at 45° C. Alternatively, hybridization to filter-bound DNA in 0.5 M NaHPO₄, 7% sodium dodecyl sulfate (SDS), 1 mM EDTA at 65° C., and washing in 0.1×SSC/0.1% SDS at 68° C. can be employed. Yet additional stringent hybridization conditions include hybridization at 60° C. or higher and 3×SSC (450 mM sodium chloride/45 mM sodium citrate) or incubation at 42° C. in a solution containing 30% formamide, 1M NaCl, 0.5% sodium sarcosine, 50 mM MES, pH 6.5. Those of ordinary skill will readily recognize that alternative but comparable hybridization and wash conditions can be utilized to provide conditions of similar stringency.

In certain embodiments, the stringency of the wash conditions that set forth the conditions that determine whether a nucleic acid is specifically hybridized to a surface bound nucleic acid. Wash conditions used to identify nucleic acids may include, e.g.: a salt concentration of about 0.02 molar at pH 7 and a temperature of at least about 50° C. or about 55° C. to about 60° C.; or, a salt concentration of about 0.15 M NaCl at 72° C. for about 15 minutes; or, a salt concentration of about 0.2×SSC at a temperature of at least about 50° C. or about 55° C. to about 60° C. for about 15 to about 20 minutes; or, the hybridization complex is washed twice with a solution with a salt concentration of about 2×SSC containing 0.1% SDS at room temperature for 15 minutes and then washed twice by 0.1×SSC containing 0.1% SDS at 68° C. for 15 minutes; or, equivalent conditions. Stringent conditions for washing can also be, e.g., 0.2×SSC/0.1% SDS at 42° C.

A specific example of stringent assay conditions is rotating hybridization at 65° C. in a salt based hybridization buffer with a total monovalent cation concentration of 1.5 M (e.g., as described in U.S. patent application Ser. No. 09/655,482 filed on Sep. 5, 2000, the disclosure of which is herein incorporated by reference) followed by washes of 0.5×SSC and 0.1×SSC at room temperature.

In one aspect, within-feature background probes comprise fewer base pairs than the specific hybridization probes. For example, in one aspect, a specific hybridization probe comprises about 20-100 base pairs, while a background probe comprises less than about 20 base pairs. In one aspect, the specific hybridization probe comprises greater than about 20 base pairs, greater than about 25 base pairs, greater than about 30, about 40, about 50, or about 60 base pairs, while the background probe comprises less than about 20 base pairs, less than about 15 base pairs, less than about 10 base pairs. In one aspect, a feature comprises about a specific hybridization probe comprising about 60 base pairs and a background hybridization probe comprising about 10 base pairs. Although background probes generally comprise the same length, in one aspect, there is variation in the length of background probes, though on average background probes are shorter than specific hybridization probes.

The proportion of biopolymers at a feature comprising background probes may vary. In one aspect, background probes comprise less than about 50% of the probes at a feature, less than about 40%, less than about 30%, less than about 20%, less than about 10%, less than about 5%, or less than about 2% of probes at a feature. Generally, however, background probes comprise at least about 1%, at least about 2%, at least about 5%, or at least about 10% of the probes at a feature. In one aspect, background probes are clustered such that a majority of background probes reside one or more specific locations within a feature. In one aspect, a location of the feature is enriched for background probes, such that a background probe is in proximity to more background probes than specific hybridization probes. In another aspect, the majority of specific hybridization arrays are at the center of a feature while the majority of background probes are at the perimeter of the feature.

Features can have widths (that is, diameter, for a round spot) in the range from a minimum of about 1.0 μm to a maximum of about 1.0 cm. In embodiments where very small spot sizes or feature sizes are desired, material can be deposited according to the invention in small spots whose width is in the range about 1.0 μm to 1.0 mm, usually about 5.0 μm to 500 μm, and more usually about 10 μm to 200 μm. Features that are not round may have areas equivalent to the area ranges of round features resulting from the foregoing diameter ranges. While the amount of specific hybridization probes and background probes within a feature may vary, in one aspect, the width of an area of the feature comprising specific hybridization probes comprises at least about 2.5 μm, at least about 5 μm, at least about 10μ, or at least about 20 μm. Generally, the width of the area is sufficiently large for target-probe binding complexes to be detected by a detector, such as an array scanner, which interrogates or reads signals (e.g., fluorescent intensity, electrical signals, and the like) from the array.

In one aspect, a within-feature background probe comprises a sequence that is a subsequence of the sequence of specific hybridization probes at that feature. For example, the specific hybridization probe comprises a sequence that is identical to the background sequence and a target identifying sequence that is substantially complementary to a target sequence to be identified to bind to that sequence under stringent hybridization conditions. In another aspect, the sequence of the within-feature background probe does not prevent binding of target to specific hybridization probes at that feature.

In one aspect, the background probe comprises one or more of: a sequence which is shorter than the sequence of the specific hybridization probe; a sequence which is empirically observed to be an inactive probe (i.e., not capable of selectively identifying target sequences under the conditions that the specific hybridization probe is capable of selectively recognizing target sequences); a sequence comprising one or more nucleotide differences from the sequence of the specific hybridization probe, a sequence capable of forming a stable intramolecular structure (e.g., such as a hairpin); a sequence comprising reverse polarity nucleotide analogs (See, e.g., U.S. Pat. Nos. 5,399,676; 5,527,899 and 5,721,218 and Koga, M. et al. (1991) J. Org. Chem. 56:3757-3759); a sequence comprising abasic phosphodiesters or modified nucleotidic units; and combinations thereof.

In one aspect, where the background probe comprises a sequence that forms a stable intramolecular structure, the specific hybridization probe comprises a destabilizing sequence that prevents or reduces the likelihood that the structure will form in the presence of a target nucleic acid.

In another aspect, the specific hybridization probe comprises background hybridization probe sequences at its 3′ terminus.

The sequence of the background probe may vary and may include repetitive or non-repetitive sequences. In one aspect, background probes sequences comprise sequences selected not to bind to any significant degree to target sequences from a particular organism being evaluated. For example, in an array of probe sequences selected or designed to evaluate human samples, background probe sequences may be obtained from bacterial or plant sequences. Sequences may be natural or synthetic or some combination thereof. Background probe sequences may be identified in silico based on the expected sequences in a target sample (e.g., human nucleic acid sequences) or may be identified by empirical testing or by using a combination of both methods.

In one aspect, within-feature background probes present in a plurality of features on the array comprise identical sequences. For example, while the specific hybridization sequences at the plurality of probes may differ, the background probes comprise identical sequences. Thus, specific hybridization probes may comprise identical background sequences but different target identifying sequences.

A variety of nucleic acid molecules can be used to form the specific hybridization probes. See, generally, Wetmur, J. (1991) Crit Rev Biochem and Mol Bio 26:227. Hybridization probes can be provided that hybridize with a variety of nucleic acid targets, such as viral, prokaryotic, and eukaryotic targets. The target may be a DNA target such as a gene (e.g., oncogene), control element (e.g., promoter, repressor, or enhancer), or sequence coding for ribosomal RNA, transfer RNA, mRNA, or RNase P. The target may be a viral genome or complementary copy thereof. Additionally, the target may be a “nucleic acid amplification product,” e.g., a nucleic acid molecule, either DNA or RNA, resulting from the introduction of an enzyme or enzymes into the cell, wherein such enzymes make a nucleic acid molecule complementary to one already present in the cell. See, e.g., O. Bagasra et al. (1992) The New England Journal of Medicine 326:1385-1391.

In certain aspects, modified nucleic acids, e.g., oligonucleotides, can be used to increase selectivity and sensitivity of the specific hybridization probes. Such modified nucleic acids are well known in the art and described in e.g., Chollet et al. (1988) Nucleic Acids Res 16:305; Potapov et al. (1996) Pure & Appl. Chem 68:1315; Soloman et al. (1993) J Org Chem 58:2232; Prosnyak et al. (1994) Genomics 21:490; Lin et al. (1991) Nucleosides & Nucleotides 10:675. For example, substitution of 2-aminoadenine for adenine, or substitution of 5-methylycytocine for cytosine can increase duplex stability. Prosnyak et al, supra. In addition, nucleic acid probes containing both types of modified bases have increased duplex stability relative to unmodified analogs. Furthermore, substitution of 2-aminoadenine (2-AA) for adenine creates an additional hydrogen bond in the Watson-Crick base pair (Chollet et al., supra), and oligonucleotide probes containing 2-AA show increased selectivity and hybridization to target DNA. In this regard, 2-AA is used only as a substitute for adenine, and binds in a manner similar to the natural base. Other examples of modified nucleic acids include the use of a base pair wherein a modified pyridone or quinolone base pairs with 2-aminopurine (Solomon et al., supra), and the use of deoxycitidine derivatives in triplex formation (Huang et al. (1996) Nucleic Acids Res. 14:2606).

In one aspect, arrays comprising within-feature background probes include validated background probes. As used herein, a “validated background probe” refers to a background probe that has been standardized/validated against a specific hybridization probes, i.e., the background probe, when bound to target, produces a known amount of signal which is less then the amount of signal produced by binding of the target to the specific hybridization probe (preferably, less than about half as much, less than about 25% as much, less than about 10% as much or less than about 1% as much of the signal produced when a target sequence binds to an appropriate target identifying sequence).

In another aspect, arrays comprise test background probes whose degree of non-specific binding to a target sample is unknown. An amount of binding of a test background probe to a target sample may be compared to an amount of binding observed for a validated background probe. In one aspect, validated background probes are included on the same substrate as test background probes. However, in some instances comparison is to an amount of binding stored in a computer memory (i.e., as data from other array-based assays).

A test background probe may be validated if the signal from the test background probe is as low as or is lower than signal from a validated background probe. In another aspect, signal replicates between test background probes must be as good as signal replicates between validated background probes in order for a test background probe to be validated.

The number of features comprising within-feature background probes as well as specific hybridization probes present on the array may vary, but is generally at least about 1, at least about 2, usually at least about 5 and more usually at least about 10, where the number of different spots on the array may be as a high as about 50, about 100, about 500, about 1000, about 10,000 or higher, depending on the intended use of the array. In one aspect, features on the array surface are generally present as a pattern, where the pattern may be in the form of organized rows and columns of spots, e.g., a grid of spots, across the substrate surface, a series of curvilinear rows across the substrate surface, e.g., a series of concentric circles or semi-circles of spots, and the like. The density of spots present on the array surface may vary, but will generally be at least about 10 and usually at least about 100 spots/cm2, where the density may be as high as 10⁶ or higher, but will generally not exceed about 10⁵ spots/cm². Nucleic acids forming features may be covalently attached to the arrays at any point along the nucleic acid chain, but are generally attached at one of their termini, e.g., the 3′ or 5′ terminus, and typically at their 3′ terminus.

The substrate may be formed of a variety of materials and the size and shape of the substrate is not a limiting feature of the invention. The substrate may be rigid or flexible or semi-flexible. The term “rigid” is used herein to refer to a structure e.g., a bottom surface that does not readily bend without breakage, i.e., the structure is not flexible. The term “flexible” is used herein to refer to a structure, e.g., a bottom surface or a cover, that is capable of being bent, folded or similarly manipulated without breakage. For example, a cover is flexible if it is capable of being peeled away from the bottom surface without breakage. In one aspect, the substrate comprises a flexible web that can be bent 180 degrees around a roller of less than 1.25 cm in radius at a temperature of 20° C. As used herein, a “web” refers to a long continuous piece of substrate material having a length greater than a width. For example, the web length to width ratio may be at least 5/1, 10/1, 50/1, 100/1, 200/1, or 500/1, or even at least 1000/1. A web substrate may be of various lengths including at least about 1 m, at least about 2 m, or at least about 5 m (or even at least about 10 m).

Rigid solid supports may be made from silicon, glass, rigid plastics, e.g. polytetrafluoroethylene, polypropylene, polystyrene, polycarbonate, etc., or metals, e.g. gold, platinum, etc. Flexible solid supports may be made from a variety of materials, such as, for example, nylon, nitrocellulose, polypropylene, polyester films, e.g., polyethylene terephthalate, polymethyl methacrylate or other acrylics, polyvinyl chloride or other vinyl resin. Various plasticizers and modifiers may be used with polymeric substrate materials to achieve selected flexibility characteristics.

Solid supports may exist in a variety of configurations ranging from simple to complex. Suitable substrates may exist, for example, as gels, sheets, tubing, spheres, containers, pads, slices, films, plates, slides, strips, plates, disks, rods, particles, beads, etc. The substrate is preferably flat, but may take on alternative surface configurations. The substrate can be a flat glass substrate, such as a conventional microscope glass slide, a cover slip and the like. Common substrates used for the arrays of probes are surface-derivatized glass or silica, or polymer membrane surfaces, as described in Guo, Z. et al. (cited above) and Maskos, U. et al., Nucleic Acids Res, 1992, 20:1679-84 and Southern, E. M. et al., Nucleic acids Res, 1994, 22:1368-73.

Immobilization of the probe to a suitable substrate may be performed using conventional techniques. See, e.g., Letsinger et al. (1975) Nucl. Acids Res. 2:773-786; Pease, A. C. et al., Proc. Nat. Acad. Sci. USA, 1994, 91:5022-5026. The surface of a substrate may be treated with an organosilane coupling agent to functionalize the surface. One exemplary organosilane coupling agent is represented by the formula RₙSiY₍₄₋ₙ₎ wherein: Y represents a hydrolyzable group, e.g., alkoxy, typically lower alkoxy, acyloxy, lower acyloxy, amine, halogen, typically chlorine, or the like; R represents a nonhydrolyzable organic radical that possesses a functionality which enables the coupling agent to bond with organic resins and polymers; and n is 1, 2 or 3, usually 1. One example of such an organosilane coupling agent is 3-glycidoxypropyltrimethoxysilane, the coupling chemistry of which is well-known in the art. See, e.g., Arkins, Silane Coupling Agent Chemistry, Petrarch Systems Register and Review, Eds. Anderson et al. (1987). Other examples of organosilane coupling agents are (γ-aminopropyl)triethoxysilane and (γ-aminopropyl)trimethoxysilane. Still other suitable coupling agents are well known to those skilled in the art. Thus, once the organosilane coupling agent has been covalently attached to the support surface, the agent may be derivatized, if necessary, to provide for surface functional groups. In this manner, support surfaces may be coated with functional groups such as amino, carboxyl, hydroxyl, epoxy, aldehyde and the like.

Use of the above-functionalized coatings on a solid support provides a means for selectively attaching oligophosphodiesters to the support. Thus, an oligonucleotide probe formed as described above may be provided with a 5′-terminal amino group, which can be reacted to form an amide bond with a surface carboxyl using carbodiimide coupling agents. 5′ attachment of the oligonucleotide may also be effected using surface hydroxyl groups activated with cyanogen bromide to react with 5′-terminal amino groups. 3′-terminal attachment of an oligonucleotide probe may be effected using, for example, a hydroxyl or protected hydroxyl surface functionality.

The subject solid supports are typically of dimensions that can be inserted into and scanned using a typically biopolymeric array scanner. Thus, subject solid supports typically have overall rectangular, square or disc configuration. In many embodiments of the subject invention, the substrate will have a rectangular shape, having a length of from about 10 mm to 200 mm, usually from about 20 to 150 mm and more usually from about 30 to 100 mm and a width of from about 5 mm to 100 mm, usually from about 10 mm to 50 mm and more usually from about 15 mm to 40 mm, and a thickness of from about 0.01 mm to 5.0 mm, usually from about 0.1 mm to 2 mm and more usually from about 0.2 to 1 mm. In one embodiment, a solid support has dimensions similar to that of a typical microscope slide, e.g., about 25 mm by 75 mm. The above dimensions are, of course, exemplary only and may vary as required.

Making Arrays Comprising Background Probes

In one embodiment, the invention provides methods for making a chemical array. The method comprises arraying a plurality of biopolymers on a substrate, thereby forming a plurality of features comprising biopolymers at known locations on the substrate. At least one feature comprises specific hybridization probes designed or selected to bind to specific target sequences and background probes for evaluating non-specific binding of target. In one embodiment, the biopolymers comprise nucleic acids.

As discussed above, arrays can be fabricated using drop deposition of either polynucleotide precursor units (such as monomers) in the case of in situ fabrication, or a previously obtained polynucleotide. Such methods are described in detail in, for example, U.S. Pat. Nos. 6,242,266, 6,232,072, 6,180,351, 6,171,797, 6,323,043, U.S. patent application Ser. No. 09/302,898 filed Apr. 30, 1999 and the references cited therein.

Drop deposition on arrays may be performed using an apparatus for delivering pulse jets. A “pulse jet apparatus” is an apparatus that can dispense drops in the formation of an array. Pulse jets operate by delivering a pulse of pressure (such as by a piezoelectric or thermoelectric element) to liquid adjacent an outlet or orifice such that a drop will be dispensed therefrom. A “drop” in reference to the dispensed liquid does not imply any particular shape, for example a “drop” dispensed by a pulse jet only refers to the volume dispensed on a single activation.

In one aspect, the method includes dispensing drops from a drop dispensing head (e.g., such as a head for dispensing pulse jets) onto a substrate while maintaining a gap between the head and substrate and moving them relative to one another (e.g., by moving either, or both, the head and substrate) along a path. Generally, the head is then moved in an opposite direction to reiterate the deposition process, with the head optionally being re-loaded with fluid between repetitions of the path.

In one aspect, the method comprises iterating a sequence of steps comprising (a) depositing drops of a protected monomer onto predetermined locations on a substrate to link with either a suitably activated substrate surface (or with previously deposited deprotected monomer); (b) deprotecting the deposited monomer so that it can react with a subsequently deposited protected monomer; and (c) depositing another protected monomer for linking. Different monomers may be deposited at different regions on the substrate during any one cycle so that the different regions of the completed array will carry the different biopolymer sequences as desired in the completed array. One or more intermediate further steps may be required in each iteration, such as oxidation and washing steps.

In one aspect, the iterative sequence is as follows: (a) coupling a selected nucleoside through a phosphite linkage to a functionalized support in the first iteration, or a nucleoside bound to the substrate (i.e. the nucleoside-modified substrate) in subsequent iterations; (b) optionally, but preferably, blocking unreacted hydroxyl groups on the substrate bound nucleoside; (c) oxidizing the phosphite linkage of step (a) to form a phosphate linkage; and (d) removing the protecting group (“deprotection”) from the now substrate bound nucleoside coupled in step (a), to generate a reactive site for the next cycle of these steps. The functionalized support (in the first cycle) or deprotected coupled nucleoside (in subsequent cycles) provides a substrate bound moiety with a linking group for forming the phosphite linkage with a next nucleoside to be coupled in step (a). Final deprotection of nucleoside bases can be accomplished using alkaline conditions such as ammonium hydroxide, in a known manner. In one aspect, a capping step is included after the oxidation step.

The foregoing chemistry of the synthesis of polynucleotides is described in detail, for example, in Caruthers, Science 230: 281-285, 1985; Itakura et al., Ann. Rev. Biochem. 53: 323-356; Hunkapillar et al., Nature 310: 105-110, 1984; and in “Synthesis of Oligonucleotide Derivatives in Design and Targeted Reaction of Oligonucleotide Derivatives”, CRC Press, Boca Raton, Fla., pages 100 et seq., U.S. Pat. Nos. 4,458,066, 4,500,707, 5,153,319, 5,869,643, EP 0294196, and elsewhere. Suitable linking layers on the substrate include those as described in U.S. Pat. Nos. 6,235,488 and 6,258,454 and the references cited therein.

In one aspect, specific hybridization probes may be formed by iteratively depositing nucleotide monomers at a location within a feature to form a biopolymer of n base pairs, while limiting biopolymer formation at another location within the feature to form a biopolymer of fewer than n base pairs.

For example, nucleotide monomers may be deposited in droplets of decreasing volume at the feature so that location(s) within a feature may be selectively exposed to monomer and synthesis can take place while other location(s) within a feature are not exposed to monomer (e.g., synthesis is limited at the other locations). Droplet volume also may be manipulated after a selected number of rounds of synthesis to control the size of within feature background probes. For example, reducing volume after 10 rounds of synthesis will result in a population of background probes enriched in 10 base pair probes.

In an alternative, or additional embodiment, the movement of a substrate or a nucleotide dispenser relative to one another (e.g., by moving the substrate, dispenser or both substrate and dispenser) may be manipulated so that a drop comprising nucleotides falls on a location of the feature to comprise specific hybridization probes and does not fall on a location of the feature to comprise background probes. In another aspect, background probes are selectively blocked after a selected number of rounds of synthesis while specific hybridization probes are not blocked so that synthesis of specific hybridization probes can proceed while synthesis of background probes is limited. Additionally, or alternatively, specific hybridization probes may be selectively activated and/or deprotected to selectively promote synthesis of specific hybridization probes. Combinations of different first and second protecting agents can be added at a feature to prevent additional monomer addition; the different agents may be selectively removed (e.g., using different conditions) so that synthesis may occur selectively at a nascent probe from which a first protecting agent has been removed while being blocked at a probe from which the second protecting agent remains. Examples of protecting groups include, but are not limited to DMT, Lev and those described in U.S. Pat. No. 6,222,030 U.S. Pat. Appl'n Publ'n No. U.S. 2002/0058802 A1, and Seio et al. (2001) Tetrahedron Lett. 42 (49):8657-8660

In still another aspect, aspect, a plurality of specific hybridization probes are synthesized and target identifying sequences are removed from a portion of these probes to generate background probes.

In a further aspect of the invention, a plurality of specific hybridization probes are synthesized having a desired length n and then blocked to prevent further synthesis. Droplet volume is then manipulated to increase the volume of the drop being deposited at the feature. Additional monomers are not added to the specific hybridization probes because these are blocked; however, new polymer synthesis occurs at the periphery of the feature. The monomer deposition process may be repeated a desired number of times until background probes of a desired length are synthesized.

Masks may be used to selectively block regions of features on the array comprising background probes while selectively exposing regions of features to comprise specific hybridization probes to nucleotides, activating agents, deprotecting agents, or combinations thereof. In one aspect, photolithography is used to selectively expose feature regions to appropriate agents during the synthesis process. Photolithographic array fabrication methods are described, for example, in U.S. Pat. Nos. 5,599,695, U.S. Pat. No. 5,753,788, and U.S. Pat. No. 6,329,143. Interfeature areas need not be present particularly when the arrays are made by photolithographic methods as described in those patents. Masks may also be used when depositing previously obtained biopolymers. For example, a location of a feature to contain background probes may be masked while depositing specific hybridization probes and a location of a feature to contain specific hybridization probes may be masked while depositing background probes.

In one embodiment, the invention additionally provides a system for making an array comprising at least one feature comprising both within-feature background probes and specific hybridization probes.

In one aspect, the system comprises a head with one or more drop dispensers (such as pulse jets), a transport system to move the head relative to the substrate, and a processor. The processor co-ordinates dispensing of droplets and movement of the drop dispensers, and may also control other movements of the head (and/or dispenser unit(s) within the head) and/or substrate. The processor also may control various functions of the dispensing unit(s) of the head as re-loading with fluid and/or nucleotides between repetitions of a scan or sweep along a path of features. Additionally, or alternatively, the processor may control characteristics of drops being dispensed, including, but not limited to: volume, velocity, viscosity, surface tension, reagents provided in a drop (e.g., synthesis reagents, such as nucleotide monomers), etc. The apparatus may also include a cutter to separate the substrate into units each of which carries at least one of the features comprising both background probes and specific hybridization probes. A printer also may be provided, which adds array identifiers to the substrate each in proximity with a corresponding array.

In a further aspect, the system comprises a substrate holder to retain a substrate thereon. Pins or similar means can be provided to align a substrate on the substrate holder; however, the substrate also may be retained on the substrate station simply by weight. In certain aspects, a vacuum chuck is provided, connectable to a suitable vacuum source for retaining a substrate on the substrate holder.

In one aspect, the system comprises a transporter for moving one or more of: the head, dispensing unit(s) within the head, and substrate holder. The operations of the transport system may be controlled by the processor. In one aspect, the substrate holder may be moved in an x-, y-, or z-direction and may be rotated or tilted. Additionally, or alternatively, the head and/or one or more dispensing units in the head may be moved in an x-, y-, or z-direction, tilted, or rotated.

In a further aspect, a dispensing unit comprises a dispensing chamber for receiving or storing a fluid (e.g., from fluid conduit(s) in communication with the dispensing unit). The dispensing unit comprises an opening or orifice communicating with the dispensing chamber, through which drops of fluid may be ejected onto a substrate positioned on the substrate holder which the orifice of the dispensing unit faces. Generally, there is a gap between the orifice and the substrate when it is place in the substrate holder. In one aspect, the system comprises an ejector in communication with fluid in the dispensing unit, which exerts a force on the fluid in the dispensing chamber in response to instructions from the processor to eject fluid from the orifice onto the substrate. The ejector may be in the form of a piezoelectric crystal operating under control of the processor; however, ejectors may also be thermally activated, activated by ultrasonic energy, mechanical energy, electrical energy and the like, and responsive to the processor through a resister. Examples of fluid ejection devices that are activated by mechanical, thermal or electrical energy include, e.g., inkjet-type devices and the like. In certain embodiments, each ejector may be in the form of an electrical resistor operating as a heating element under control of processor (although piezoelectric elements may be used instead). In this manner, application of a single electric pulse to an ejector causes a volume of fluid to be dispensed from a corresponding orifice.

In another aspect, an encoder communicates with the processor to provide data on the location of the substrate holder (and hence substrate) while another encoder provides data on the location of the head and/or dispensing units within the head. Any suitable encoder, such as an optical encoder, may be used which provides data on linear position. A head system may be employed that includes two, three or more heads which may be mounted on different holders or mounted on the same holder for movement in unison with one another (or may be mounted for independent movement).

Elements of the head and its dispensing unit(s) can be adapted from commercially available piezoelectric inkjet print heads. One type of head and other suitable dispensing head designs are described in more detail in U.S. Pat. Nos. 6,461,812; 6, 440,669; 6,323,043; 6,599,693, for example. However, other head system configurations may be used.

In one aspect, dispensing parameters of the dispensing unit are manipulated in response to instructions from the processor, to generate within-feature background probes according to the invention. For example, the diameter of an orifice of a dispensing unit of the head may be altered (e.g., by manipulating a shutter for sealing an orifice), the volume of fluid delivered by the dispensing chamber may be altered, the velocity of fluid ejected may be altered, the amount of force delivered by the ejector, also may be altered, etc.

Generally, the amount of fluid that is expelled during a single activation event is in the range about 0.1 to 1000 pL, usually about 0.5 to 500 pL and more usually about 1.0 to 250 pL. In one aspect, to limit synthesis of background probes after a selected number of rounds of synthesis, the amount of fluid is reduced by at least about 2-fold, at least about 4-fold, at least about 8 fold, or at least about 10-fold.

Additionally, or alternatively, movements of the substrate and/or head and/or dispensing unit(s) may be manipulated before or during a sweep or scan along a path on the substrate to contain features, e.g., such that the substrate is offset relative to the dispensing head to limit synthesis at a location in at least one feature.

In one aspect, the system is used in conjunction with a mask which physically blocks portions of a feature during the deposition process after a selected number of rounds of synthesis to selectively expose specific hybridization probes to nucleotides and/or activating agents and/or deprotecting agents. In another aspect, a portion of a feature comprising specific hybridization probes is blocked while another portion comprising specific hybridization probes is exposed to an agent that removes or cleaves target identifying sequences from the probes.

In one embodiment, the system further includes a sensor, e.g., such as a camera, to monitor drops dispensed by dispensing units and/or to monitor a substrate surface. See, e.g., as described in U.S. Pat. No. 6,232,072. The camera communicates with the processor and preferably has a resolution that provides a pixel size of about 1 to 100 micrometers and more typically about 4 to 20 micrometers or even 1 to 5 micrometers. Any suitable analog or digital image capture device (including a line by line scanner) can be used for such camera, although if an analog camera is used, the processor preferably includes a suitable analog/digital converter. Monitoring can occur during formation of an array and the information used during fabrication of the remainder of that array or another array, or test-print patterns can be run before array fabrication.

In another aspect the system includes one or more of: a display, speaker, and operator input device. An operator input device may, for example, be a keyboard, mouse, or the like. Any or all of these may be in communication with the processor.

In one aspect, the processor has access to a memory. The memory may be a magnetic, optical, or solid state storage device (including magnetic or optical disks or tape or RAM, or any other suitable device, either fixed or portable). The processor may include a general purpose digital microprocessor suitably programmed from a computer readable medium carrying necessary program code, to execute all of the functions required of it as described above. Suitable programming can be provided remotely to the processor, or previously saved in a computer program product such as memory or some other portable or fixed computer readable storage medium. For example, a magnetic or optical disk may carry the programming, and can be read by disk reader in communication with the processor.

In one aspect, the memory includes instructions for altering dispensing parameters and/or movements of the substrate relative to dispensing unit(s) of the head during one or more scans or sweeps along a path of a substrate to generate suitable background probes at locations within a feature.

In one embodiment, the invention provides a system for generating an array comprising a plurality of features, at least one feature comprising background probes and specific hybridization probes. The system comprises a substrate holder for retaining a substrate, a head comprising a drop dispenser comprising an opening facing a retained substrate for depositing agents for biopolymer synthesis on the retained substrate; a transport system for moving the head relative to the substrate; and a processor for controlling the ejection of drops from the drop dispenser onto the substrate according to a predetermined pattern during movement of the head relative to the substrate, thereby forming a pattern of features comprising biopolymers on the substrate. The pattern includes the formation of at least one feature comprising background probes and specific hybridization probes.

As discussed above, the pattern may be generated by providing instructions to various system components (e.g., transporter(s), ejector(s), orifice shutters, and the like). In one aspect, the pattern is dictated by programmed instructions provided to the processor, e.g., by a user of the system, remotely, from another system, and/or in response to feedback from one or more system components (e.g., such as from an encoder, and/or camera).

In another embodiment, the invention further provides a computer program product comprising a computer readable medium comprising instructions for performing any of the above-disclosed methods or system operations.

In a further embodiment, the invention provides a remote ordering system for ordering an array comprising at least one feature comprising both specific hybridization probes and background probes. In response to receiving such an order, the processor communicates instructions to the system according to the invention to produce such an array.

Methods of Using Arrays Comprising Background Probes

In one aspect, the method comprises providing an array comprising a plurality of biopolymer features arrayed at known locations on a substrate, wherein at each of the features comprises a plurality of specific hybridization probes and a plurality of background probes; detecting binding of target to a feature and determining an amount of binding at the feature; determining an amount of binding of target to background probes in the feature and adjusting the amount of binding at the feature based on the amount of binding determined for the background probe. In one aspect, the target is labeled (such as with a fluorescent label) and formation of a complex between a target and probe (whether a specific hybridization probe or background probe) results in a detectable signal (e.g., fluorescence). Background signal may be subtracted from observed signal at a feature in order to determine the presence and/or amount of target sequence in the sample, since the remaining signal should represent signal contributed by complexes between the specific hybridization probe and the target representing selective binding of the probe to the target (i.e., based on sequence characteristics of the target).

In one aspect, an array according to the invention is contacted with a fluid sample suspected of containing a target nucleotide sequence and incubated under suitable hybridization conditions. Hybridization generally takes from about 30 minutes to about 24 hours, and occurs at the highest specificity approximately 10-25° C. below the temperature (Tₘ) at which the nucleotide hybrid is 50% melted. The Tₘ for a particular hybridization pair will vary with the length and nature of the nucleotides and may be readily determined by those of ordinary skill in the art.

Generally, a nucleic acid molecule is capable of hybridizing selectively or specifically to a target sequence under moderately stringent hybridization conditions. In the context of the present invention, moderately stringent hybridization conditions generally allow detection of a target nucleic acid sequence of at least 14 nucleotides in length having at least approximately 70% sequence identity with the sequence of the selected nucleic acid probe. In another embodiment, such selective hybridization is performed under stringent hybridization conditions. Stringent hybridization conditions allow detection of target nucleic acid sequences of at least 14 nucleotides in length having a sequence identity of greater than 90% with the sequence of the selected nucleic acid probe. Hybridization conditions useful for probe/target hybridization where the probe and target have a specific degree of sequence identity, can be determined as is known in the art (see, for example, Nucleic Acid Hybridization: A Practical Approach, editors B. D. Hames and S. J. Higgins, (1985) Oxford; Washington, D.C.; IRL Press) and as discussed above.

With respect to stringency conditions for hybridization, it is well known in the art that numerous equivalent conditions can be employed to establish a particular stringency by varying, for example, the following factors: the length and nature of probe and target sequences, base composition of the various sequences, concentrations of salts and other hybridization solution components, the presence or absence of blocking agents in the hybridization solutions (e.g., formamide, dextran sulfate, and polyethylene glycol), hybridization reaction temperature and time parameters, as well as, varying wash conditions. The selection of a particular set of hybridization conditions is well within the skill of a person of ordinary skill in the art (see, for example, Sambrook, et al., Molecular Cloning: A Laboratory Manual, Second Edition, (1989) Cold Spring Harbor, N.Y.).

In general, hybridization is carried out in a buffered aqueous medium typically formulated with a salt buffer, detergents, nuclease inhibitors and chelating agents, using techniques well-known to those skilled in the art. Such formulations may be selected to preclude significant nonspecific binding of nucleotides with the support-bound array. Various solvents may be added to the medium such as formamide, dimethylformamide and dimethylsulfoxide, and the stringency of the hybridization medium may be controlled by temperature, pH, salt concentration, solvent system, or the like. Defining appropriate hybridization conditions is within the skill of the art. See, e.g., Sambrook, Fritsch & Maniatis, Molecular Cloning: A Laboratory Manual, Second Edition (1989). In one aspect, prior to the detection step, any target nucleic acid present in the initial sample contacted with the array is labeled with a detectable label. Labeling can occur either prior to or following contact with the array. In other words, the nucleic acids present in the fluid sample contacted with the array may be labeled prior to or after contact, e.g., hybridization, with the array. In some embodiments, the sample nucleic acids (including the target nucleotide sequence(s) if present in the sample) are directly labeled with a detectable label, wherein the label may be covalently or non-covalently attached to the nucleic acids of the sample. For example, the nucleic acids, including the target nucleotide sequence, may be labeled with biotin, exposed to hybridization conditions, wherein the labeled target nucleotide sequence binds to an avidin-label or an avidin-generating species. In an alternative embodiment, the target nucleotide sequence is indirectly labeled with a detectable label, wherein the label may be covalently or non-covalently attached to the target nucleotide sequence. For example, the label may be non-covalently attached to a linker group, which in turn is (i) covalently attached to the target nucleotide sequence, or (ii) comprises a sequence which is complementary to the target nucleotide sequence. In another example, the probes may be extended, after hybridization, using chain-extension technology or sandwich-assay technology to generate a detectable signal (see, e.g., U.S. Pat. No. 5,200,314). Generally, such detectable labels include, but are not limited to, radioactive isotopes, fluorescers, chemiluminescers, enzymes, enzyme substrates, enzyme cofactors, enzyme inhibitors, dyes, metal ions, metal sols, ligands (e.g., biotin or haptens) and the like. A variety of biological and/or chemical compounds may be used as detectable labels in the above-described arrays (See, e.g., Wetmur, J. Crit Rev Biochem and Mol Bio 26:227, 1991; Mansfield et al., Mol Cell Probes. 9:145-56, 1995; Kricka, Ann Clin Biochem. 39:114-29, 2002).

In one embodiment, the label is a fluorescent compound, i.e., capable of emitting radiation (visible or invisible) upon stimulation by radiation of a wavelength different from that of the emitted radiation, or through other manners of excitation, e.g. chemical or non-radiative energy transfer. The label may be a fluorescent dye. Preferably, a target with a fluorescent label includes a fluorescent group covalently attached to a nucleic acid molecule capable of binding specifically to the complementary probe nucleotide sequence. Fluorescent groups useful as labels in this invention include, but are not limited to, fluorescein (or FITC), Texas Red, coumarin, rhodamine, rhodamine derivatives, phycoerythrin, Perci-P, 4-methylumbelliferyl phosphate, resorufin, 7-diethylamino coumarin-3-carboxylic acid succinimidyl ester, and the like. Fluorescent groups having near infrared fluorescence include, but are not limited to, indocyanine green [CAS 3599-32-4], copper phthalocyanine [CAS 147-14-8], 3,3′-diethyl-19,11:15,17-dienopentylene-2,2′-thiapentacarbocyanine, and the like.

Additionally, the label may be an aromatic compound (having one or more benzene or heteroaromatic rings or polycyclic aromatic or heteroaromatic structures). Labels for use in the present invention may also include chemiluminescent groups such as, but are not limited to, isoluminol (4-aminophthalhydrazide), and the like. In an additional embodiment, the label is a protein or an enzyme. In a preferred embodiment, the enzyme is capable of catalyzing a reaction that produces a detectably labeled product

Methods for attaching labels to target nucleotide sequence are similar to the methods for attaching labels to probes which are well known in the art. (See e.g., U.S. Pat. Nos. 5,260,433; 5,241,060; 4,994,373; 5,401,837 and 5,141,183). For example, a primary amine can be attached to a 3′ oligo terminus or a 5′ oligo terminus. The amines can be reacted to various haptens using conventional activation and linking chemistries. International Publication Nos. WO 92/10505 and WO 92/11388 teach methods for labeling polynucleotides at their 5′ and 3′ ends, respectively. According to one known method for labeling an oligonucleotide, a label-phosphoramidite reagent is prepared and used to add the label to the oligonucleotide during its synthesis. See, for example, N. T. Thuong et al. (1988) Tet. Letters 29:5905-5908. Preferably, target polynucleotides are labeled multiple times by inclusion of labeled nucleotides during target oligonucleotide synthesis.

Following hybridization and labeling, as described above, the label is detected using colorimetric, fluorimetric, chemiluminescent or bioluminescent means. Fluorescent labels are detected by allowing the fluorescent molecule to absorb energy and then emit some of the absorbed energy; the emitted energy is then detected using fluorimetric means. In one aspect, the fluorescent dye is excitable by inexpensive commercially available lasers (e.g. HeNe, Micro Green, or solid state), has a quantum yield greater than 10%, exhibits low photo-bleaching and can be easily incorporated into target. In a preferred embodiment, when the target is labeled with R6G (Rhodamine-6-G), the label is detected by exciting at about 480 nm to about 550 nm, preferably at about 524 nm, and measuring light emitted at wavelengths at about 530 nm to about 610 nm, preferably at about 557 nm. Generally, reasonable precautions are taken to minimize the concentration of species that absorb the excitation energy and emit in the detection range.

Chemiluminescent label groups are detected by allowing them to enter into a reaction, e.g., an enzymatic reaction, that results in the emission of energy in the form of light. Other labels, e.g. biotin, may be detected because they can bind to groups such as streptavidin which are bound, directly or indirectly to enzymes, e.g. (alkaline phosphatase or horseradish peroxidase) that can catalyze a detectable reaction.

The signals detected from the hybridization features are then background corrected with signals obtained from the hybridization features in order to obtain background corrected signals for each hybridization feature of interest on the array. The background corrected signals are generally obtained by subtracting the signal of a background feature, or the average signal from a plurality of background features.

The resultant background corrected signals for each of the hybridization features are then employed to detect the presence of the analyte of interest in the assayed sample, either qualitatively or quantitatively.

In certain embodiments, the subject methods include a step of transmitting data from at least one of the detecting and deriving steps, as described above, to a remote location. By “remote location” is meant a location other than the location at which the array is present and hybridization occur. For example, a remote location could be another location (e.g. office, lab, etc.) in the same city, another location in a different city, another location in a different state, another location in a different country, etc. The data may be transmitted to the remote location for further evaluation and/or use. Any convenient telecommunications means may be employed for transmitting the data, e.g., facsimile, modem, internet, etc.

In another embodiment, the invention provides a method for evaluating data from an array comprising a plurality of features, at least one feature comprising both specific hybridization probes and background probes. Labeled biological sample(s) (i.e., “target”) are then prepared, labeled and hybridized to probes on the array.

Typically, radioactivity or some form of electromagnetic energy is used to measure responses at each probe. For example, a scanner may be used to read the fluorescence of resultant surface bound molecules under illumination with suitable (most often laser) light. The scanner acts like a large field fluorescence microscope in which the fluorescent pattern caused by binding of labeled molecules is scanned on the substrate. In particular, a laser induced fluorescence scanner may be used to analyze large numbers of different target molecules of interest, e.g., genes/mutations/alleles, in a biological sample.

The scanning equipment typically used for the evaluation of microarrays includes a scanning fluorometer. A number of different types of such devices are commercially available from different sources, such as Axon Instruments in Union City, Calif.; Perkin Elmer of Wellesly, Mass.; and Agilent Technologies, Inc. of Palo Alto, Calif. Analysis of the data, (i.e., collection, reconstruction of image, comparison and interpretation of data) is performed with associated computer systems and commercially available software, such as GenePix by Axon Instruments, QuantArray by Perkin Elmer, Feature Extraction by Agilent of Palo Alto, Calif., or Affy Scanner, available from Affymetrix, Santa Clara, Calif. Reading methods may include other optical techniques (for example, detecting chemiluminescent or electroluminescent labels) or electrical techniques (where each feature is provided with an electrode to detect hybridization at that feature in a manner disclosed in U.S. Pat. No. 6,251,685, U.S. Pat. No. 6,221,583 and elsewhere).

In one aspect, quantitative evaluation of a feature comprises the step of predicting the geometry and dimensions of a feature comprising both specific hybridization probes and background probes. For example, a template, analogous to a cookie cutter, may be positioned within each area of an array or image of an array that is predicted to have a feature deposited or written thereon. In one aspect, the template conforms to the dimensions of a location of a feature expected to comprise background probes (e.g., such as the perimeter of a feature, when backround probes are synthesized or limited to the perimeter of the feature and specific hybridization probes are synthesized at the center of the feature) and a signal corresponding to background signal is extracted for subtraction from the total signal at the feature.

On two color (two channel) systems, direct comparisons are optimal between two different biological samples, wherein one sample is encoded with a green fluorescing dye and the other is encoded with a red fluorescing dye, for example. The differential gene expression between the two samples is then given by the color at each probe because the color is determined by how much red fluorescence and green fluorescence is present at each probe. With a one color, or single channel system, absolute signals or intensities are measured. With a single channel system, one biological sample may be measured on a microarray, and a second biological sample can be measured on a second microarray. The readings are then compared to determine ratios between the results of the two arrays. In one aspect, readings on both channels are corrected to account for non-specific binding of target molecules, e.g., by adjusting by an amount determined from measuring signal at a location of a feature comprising background probes.

Scanner output may be represented as an image file of ordered sequential signals (such as a TIFF file, for example). Image processing is then performed to organize signal patterns and quantitate the value at each feature (localized probe or “spot”), or to evaluate the values of red and green at each feature for a two-channel system. Once the features values are determined, ratios can be calculated. A result obtained from the reading followed by a method of the present invention may be used in that form or may be further processed to generate a result such as that obtained by forming conclusions based on the pattern read from the array (such as whether or not a particular target sequence may have been present in the sample, or whether or not a pattern indicates a particular condition of an organism from which the sample came). A result of the reading (whether further processed or not) may be forwarded (such as by communication) to a remote location if desired, and received there for further use (such as further processing).

Specific target detection applications of interest include hybridization assays in which the nucleic acid arrays of the subject invention are employed. In these assays, a sample of target nucleic acids is first prepared, where preparation may include labeling of the target nucleic acids with a label, e.g., a member of signal producing system. Following sample preparation, the sample is contacted with the array under hybridization conditions, whereby complexes are formed between target nucleic acids that are complementary to probe sequences attached to the array surface. The presence of hybridized complexes is then detected. Specific hybridization assays of interest include, but are not limited to: gene discovery assays, differential gene expression analysis assays; nucleic acid sequencing assays, and the like. Patents and patent applications describing methods of using arrays in various applications include: 5,143,854; 5,288,644; 5,324,633; 5,432,049; 5,470,710; 5,492,806; 5,503,980; 5,510,270; 5,525,464; 5,547,839; 5,580,732; 5,661,028; 5,800,992; WO 95/21265; WO 96/31622; WO 97/10365; WO 97/27317; EP 373 203; and EP 785 280.

Kits

Kits for use in target detection assays are provided. The subject kits at least include the arrays of the subject invention. The kits may further include one or more additional components necessary for carrying out the analyte detection assay, such as sample preparation reagents, buffers, labels, and the like. As such, the kits may include one or more containers such as vials or bottles, with each container containing a separate component for the assay, such as an array, and reagents for carrying out nucleic acid hybridization assays according to the invention. The kit may also include a denaturation reagent for denaturing target molecules, hybridization buffers, wash solutions, enzyme substrates, negative and positive controls and written instructions for carrying out the assay. In addition, the kits typically further include instructions for how practice the subject analyte detection methods according to the subject invention, where these instructions are generally present on at least one of a package insert and the package of the kit. In one aspect, the kit comprises one or more computer program products according to the invention.

What is claimed is:

1. A method for making a chemical array comprising:
arraying a plurality of biopolymers on a substrate, thereby forming a plurality of features comprising biopolymers at known locations on the substrate,
wherein at least one feature comprises specific hybridization probes designed or selected to specifically bind to target sequences and background probes for evaluating non-specific binding of target.

2. The method of claim 1, wherein biopolymers forming features are synthesized in situ on the substrate.

3. The method of claim 1, wherein the biopolymers comprise nucleic acids.

4. The method of claim 1, wherein the background probes are located at the perimeter of the feature.

5. The method of claim 4, wherein the specific hybridization probes are located at the center of the feature.

6. The method of claim 1, wherein the specific hybridization probes are on average about 60 base pairs or greater.

7. The method of claim 1, wherein the specific hybridization probes are on average less than about 60 base pairs.

8. The method of claim 1, wherein the background probes comprise fewer base pairs than the specific hybridization probes.

9. The method of claim 1, wherein the background probes comprise at least about 5 base pairs.

10. The method of claim 1, wherein the background probes comprise at least about 10 base pairs.

11. The method of claim 1, wherein specific hybridization probes are formed by iteratively depositing nucleotide monomers at a selected location within a feature to form a biopolymer.

12. The method of claim 1, wherein specific hybridization probes are formed by iteratively depositing nucleotide monomers at a location within a feature to form a biopolymer of n base pairs, while limiting biopolymer formation at another location within the feature to form a biopolymer of fewer than n base pairs.

13. The method of claim 12, wherein nucleotide monomers are deposited in droplets of decreasing volume at the feature.

14. The method of claim 12, wherein nucleotide monomers are deposited in droplets by a droplet dispenser and either or both the droplet dispenser or substrate is moved so that the droplet falls on a location of the feature to comprise specific hybridization probes and does not fall on a location of the feature to comprise background probes.

15. The method of claim 12, wherein the background probes are selectively blocked while nucleotide monomers are deposited.

16. The method of claim 12, wherein, specific hybridization probes are selectively activated while nucleotide monomers are deposited.

17. A chemical array formed by the method of claim 1.

18. A chemical array comprising a plurality of features, wherein each feature comprises a plurality of specific hybridization probes and background probes, and wherein the background probe sequences comprise a subsequence of the specific hybridization probes and background probes within at least two features are identical.

19. The array of claim 18, wherein each feature comprises substantially identical background probes.

20. A substrate comprising a plurality of chemical arrays, wherein one of the plurality of arrays is an array according to claim 18.

21. A method for detecting specific binding of a target molecule in a sample to a probe comprising:
providing an array comprising a plurality of biopolymer features arrayed at known locations on a substrate, wherein at least one feature comprises a plurality of specific hybridization probes and a plurality of background probes;
detecting binding of target to a feature and determining an amount of binding at the feature;
determining an amount of binding of target to background probes in the feature to determine an amount of non-specific binding.

22. The method of claim 19, wherein the target is labeled with a fluorescent label and the detecting step comprises determining intensity of fluorescence at the feature.

23. The method of claim 19, wherein each feature comprises background probes comprising less than about 20 base pairs in length.

24. The method of claim 19, wherein the specific hybridization probes are about 60 base pairs in length.

25. The method of claim 19, wherein background probes are about 10 base pairs in length.

26. The method of claim 19, wherein the specific hybridization probes are about 25 base pairs in length.

27. A system for performing a method according to claim 1, comprising:
a substrate holder for retaining a substrate;
a head comprising a drop dispenser comprising an opening facing a retained substrate for depositing agents for biopolymer synthesis on the retained substrate;
a transport system for moving the head and/or drop dispenser relative to the substrate; and
a processor for controlling the ejection of drops from the drop dispenser onto the substrate according to a predetermined pattern during movement of the head and/or drop dispenser relative to the substrate, thereby forming a pattern of features comprising biopolymers on the substrate,
wherein the pattern includes formation of at least one feature comprising background probes and specific hybridization probes.

28. The system of claim 27, wherein the processor provides instructions for changing the volume of a drop ejected from the drop dispenser after a selected number of passes of the drop dispenser over a location on the substrate.

29. The system of claim 27, wherein the processor offsets the position of the drop dispenser relative to a location receiving previously deposited drops.

30. A computer program product comprising instructions for forming a pattern of features on an array using a system according to claim 27, at least one feature comprising specific hybridization probes designed or selected to specifically bind to target sequences and background probes for evaluating non-specific binding of target.

31. A method for evaluating non-specific background signal at a feature on a chemical array which comprises background probes and specific hybridization probes, comprising:
positioning a template within an area of an array or an image of an array that is predicted to have a feature deposited or written thereon, wherein the template conforms in dimensions to an area of a feature comprising background probes, and excludes an area of the feature comprising specific hybridization probes; and
extracting feature information within the template area.

32. A method comprising:
receiving data for a feature on a chemical array comprising both specific hybridization probes designed or selected to specifically bind to target sequences and background probes for evaluating non-specific binding of target; and
detecting an amount of specific hybridization to the target sequences.

33. A method for ordering a chemical array comprising at least one feature comprising background probes and specific hybridization probes, comprising providing instructions to the processor of the system of claim 27, specifying the predetermined pattern.

34. The method of claim 33, wherein the instructions comprise instructions relating to the sequence of the background probes and/or the specific hybridization probes.

35. The method of claim 33, wherein the instructions comprise instructions relating to the location of features on the substrate.
